(19) United States
(12) Patent Application Publication
(10) Pub. No.: US 2013/0280189 A1
(43) Pub. Date: Oct. 24, 2013

(54) PERSONAL CARE AND HEALTH CARE COMPOSITIONS

(71) Applicants: Xavier Jean-Paul Thomas, Famars (FR); Timothy Mitchell, Clio, MI (US)

(72) Inventors: Xavier Jean-Paul Thomas, Famars (FR); Timothy Mitchell, Clio, MI (US)

(73) Assignees: DOW CORNING FRANCE SAS, Lyon (FR); DOW CORNING CORPORATION, Midland, MI (US)

(21) Appl. No.: 13/994,864

(22) PCT Filed: Jan. 9, 2012
(86) PCT No.: PCT/IB12/00249

§ 371 Date: Jul. 2, 2013

Related U.S. Application Data
(60) Provisional application No. 61/431,165, filed on Jan. 10, 2011.

(51) Int. Cl. A61K 8/895; A61K 31/80

(52) U.S. Cl. CPC A61K 8/895; A61K 31/80
USPC 424/59; 424/78.02; 424/70.12; 424/61; 424/62

(57) ABSTRACT

The present invention relates to topical compositions comprising a non adhesive film forming silicone acrylate hybrid composition, at least one physiologically acceptable ingredient and at least one physiologically acceptable solvent. Such non adhesive film forming silicone acrylate hybrid compositions tend to provide comfortable films for topical compositions.

### CROSS-REFERENCE TO RELATED APPLICATIONS

This application claims the benefit of U.S. provisional patent application No. 61/431,165 filed Jan. 10, 2011.

### BACKGROUND OF THE INVENTION

Disclosed herein are topical compositions comprising a non adhesive film forming silicone acrylate hybrid composition and at least one physiologically acceptable ingredient.

Silicones may be present in topical compositions to form a film on skin, to solubilise an active ingredient, or to modify the texture of the composition.

Silicone pressure sensitive adhesive, having adhesive properties, may be used as drug delivery systems.

The non adhesive film forming silicone acrylate hybrid composition disclosed herein provides comfortable films for topical compositions.

### BRIEF SUMMARY OF THE INVENTION

Disclosed herein are topical compositions comprising a non adhesive film forming silicone acrylate hybrid composition in a physiologically acceptable medium. Such compositions tend to provide desirable characteristics for use in topical applications such as long lastingness, barrier property, drug delivery.

### DETAILED DESCRIPTION OF THE INVENTION

Pressure sensitive adhesive silicone acrylate hybrid compositions have been described in patent application PCT/US2007/013321 filed on Jun. 6, 2007, the disclosure of which is hereby incorporated by reference.

The silicone acrylate hybrid composition is the reaction product of (I) a silicon-containing pressure sensitive adhesive that contains acrylate or methacrylate functionality, (II) an ethylenically unsaturated monomer, and (III) an initiator. As used herein, the term silicone acrylate denotes a polymerized hybrid species that includes silicone-based sub-species and acrylate-based sub-species that have been polymerized together.

The silicon-containing pressure sensitive adhesive (I) comprises acrylate or methacrylate functionality. The silicon-containing pressure sensitive adhesive (I) may include only acrylate functionality, only methacrylate functionality, or both acrylate functionality and methacrylate functionality. The silicon-containing pressure sensitive adhesive is present in the hybrid composition in an amount of from 5 to 95, alternatively 25 to 75, parts by weight based on 100 parts by weight of the hybrid composition.

The silicon-containing pressure sensitive adhesive (I) is typically produced by the condensation reaction product of (i) a pressure sensitive adhesive and (ii) a silicon-containing capping agent wherein the capping agent provides the acrylate or methacrylate functionality to the silicon-containing pressure sensitive adhesive (I).

The pressure sensitive adhesive (i) comprises the condensation reaction product of (a) a silicone resin and (b) a polydiorganosiloxane. Although not required, the pressure sensitive adhesive (i) may comprise a catalytic amount of a condensation catalyst.

There is a wide array of silicone resins (a) and polydiorganosiloxanes (b) that are suitable to make up the pressure sensitive adhesive (i). Suitable silicone resins (a) and polydiorganosiloxanes (b) include, but are not limited to, those disclosed and described in U.S. Pat. No. 6,337,086 to Kanios et al., the disclosure of which is incorporated by reference herein in its entirety.

A typical silicone resin (a) comprises triorganosiloxy units of the formula R³₃SiO_{1/2} and tetrafunctional siloxy units of the formula SiO_{4/2} in a ratio of about 0.6 to 0.9 triorganosiloxy units for each tetrafunctional siloxy unit, wherein each R³ independently denotes a monovalent hydrocarbon radical having from 1 to 6 carbon atoms such as methyl, ethyl, propyl, isopropyl, hexyl, hexenyl, cyclohexyl, vinyl, allyl, propenyl and phenyl.

There may also be a few mole percent of R³₂SiO units present in the silicone resin (a).

Silicone resin (a) may be prepared according to Daudt et al., U.S. Pat. No. 2,676,182 (issued Apr. 20, 1954 and hereby incorporated by reference) whereby a silica hydrosol is treated at a low pH with a source of R³₃SiO_{1/2} units such as a hexaorganodisiloxane such as Me₃SiOSiMe₃, ViMe₂SiOSiMe₂Vi or MeViPhSiOSiPhViMe or triorganosilane such as Me₃SiCl, Me₂ViSiCl or MeViPhSiCl.

The silicone resins (a) contain silicon-bonded hydroxyl radicals in amounts which typically range from about 1 to 4 weight percent of silicon-bonded hydroxyl radicals. There should be at least some and alternatively at least 0.5% silicon-bonded hydroxyl content to enable the polydiorganosiloxane (b) to copolymerize with the silicone resin (a). The silicon-bonded hydroxyl radicals may also react with the endblocking agent being added to chemically treat the pressure sensitive adhesive (i).

The silicone resins (a) are generally benzene-soluble resinous materials which are typically solids at room temperature and are prepared as, and usually, but not necessarily used as, a solution in an organic solvent or cosmetic solvent. Typical organic solvents used to dissolve silicone resins (a) include benzene, toluene, xylene, methylene chloride, perchloroethylene, naphtha mineral spirits and mixtures of these. Typical cosmetic solvents used to dissolve silicone resins (a) include cyclomethicone, dimethicone, ethanol, isopropyl alcohol, mineral oil, sunflower oil, caprylic/capric triglyceride.

A typical polydiorganosiloxane (b) comprises AR³SiO units with terminal TR³ASiO_{1/2} units, where each A radical is independently selected from R³ or halohydrocarbon radicals having from 1 to 6 carbon atoms such a chloromethyl, chloropropyl, 1-chloro-2-methylpropyl, 3,3,3-trifluoropropyl and F₃C(CH₂)₅ radicals, each T radical is independently selected from the group consisting of R³, OH, H or OR⁴, and each R⁴ is independently an alkyl radical having from 1 to 4 carbon atoms such as methyl, ethyl, n-propyl, and isobutyl radicals.

Polydiorganosiloxane (b) has a viscosity of from 100 centipoise to 30,000,000 centipoise at 25° C. Polydiorganosiloxanes (b) having a viscosity of from about 100 to 100,000 centipoise at 25° C. range from fluids to somewhat viscous polymers. These polydiorganosiloxanes (b) may be prereacted with silicone resin (a) prior to condensation in the presence of an endblocking agent. Polydiorganosiloxanes (b) having viscosities in excess of 100,000 centipoise may typically be subjected to the condensation/endblocking step without prereaction. Polydiorganosiloxanes (b) having viscosities in excess of 1,000,000 centipoise are highly viscous products often referred to as gums and the viscosity is often expressed in terms of a Williams Plasticity value (polydimethylsiloxane gums of about 10,000,000 centipoise viscosity typically have a Williams Plasticity Value of about 1.27 mm (50 mils) or more at 25° C.).

Polydiorganosiloxane (b) typically has the formula AR³SiO such as Me₂SiO units, PhMeSiO units, MeViSiO units, Ph₂SiO units, methylethylsiloxy units, 3,3,3-trifluoropropyl units and 1-chloro, 2-methylpropyl units and the like.

Alternatively, the AR³SiO units are selected from the group consisting of R³₂SiOR³R⁴SiO units, Ph₂SiO units and combinations of both. At least 50 mole percent of the R⁴ radicals present in the polydiorganosiloxane (b) are methyl radicals and no more than 50 mole percent of the total moles of AR³SiO units present in each polydiorganosiloxane (b) are Ph₂SiO units. Alternatively, no more than 10 mole percent of the AR³SiO units present in each polydiorganosiloxane (b) are MeR³SiO units and the remaining AR³SiO units present in each polydiorganosiloxane (b) are Me₂SiO units. Alternatively, substantially all of the AR³SiO units are Me₂SiO units.

The H, OH and OR⁴ in the terminal units of polydiorganosiloxane (b) provide a site for reaction with the acrylate or methacrylate functional silicon-containing capping agent (ii) and also provide a site for condensation with other such radicals on polydiorganosiloxanes (b) or with the silicon-bonded hydroxyl groups present in silicone resin (a). Use of polydiorganosiloxanes (b) where T is OH is appropriate because the polydiorganosiloxane (b) may then readily copolymerize with the silicone resin (a). When an appropriate catalyst such as HCl, which is generated when chlorosilanes are used, or ammonia, which is generated when organosilazanes are used as endblocking agents, then triorganosiloxy (e.g., R³₃SiO_{1/2} such as (CH₃)₃SiO_{1/2} or CH₂CH(CH₃)₂ SiO_{1/2}) unit terminated polydiorganosiloxanes (b) may be employed because some of the triorganosiloxy units may be cleaved when the condensation reaction is conducted with heating. The cleavage exposes a silicon-bonded hydroxyl radical which may then condense with silicon-bonded hydroxyl radicals in the silicone resin (a), with endblocking triorganosilyl units or with other polydiorganosiloxanes (b) containing H, OH or OR⁴ radicals or silicon-bonded hydroxyl radicals exposed by cleavage reactions. Mixtures of polydiorganosiloxanes (b) containing different substituent radicals may also be used.

When pressure sensitive adhesives (i) are to be cured by peroxide or through aliphatically unsaturated radicals present in silicone resin (a) or polydiorganosiloxane (b), if silicone resin (a) contains aliphatically unsaturated radicals, then polydiorganosiloxane (b) should be free of such radicals and vice-versa. If both components contain aliphatically unsaturated radicals, curing through such radicals may result in products which do not act as pressure sensitive adhesives.

The pressure sensitive adhesives (i) are made using from 30 to 80, alternatively 40 to 75, parts by weight of silicone resins (a) and from 20 to 70, alternatively 25 to 60, parts by weight of polydiorganosiloxane (b). Although not required, the pressure sensitive adhesive (i) may comprise a catalytic amount of a condensation catalyst.

The pressure sensitive adhesive (i), comprising silicon bonded hydroxyl groups (i.e., silanols), and the silicon-containing capping agent (ii) are condensed to produce the silicon-containing pressure sensitive adhesive (I). Once the silicon-containing capping agent (ii) reacts with the pressure sensitive adhesive (i), the concentration of silanols in the silicon-containing pressure sensitive adhesive (I) is typically from 5,000 to 15,000, more typically from 8,000 to 13,000 ppm.

The pressure sensitive adhesive (i) is present in the silicon-containing pressure sensitive adhesive (I) in an amount of from 85.0 to 99.9, alternatively 90.0 to 99.8, alternatively 50 to 65, alternatively 60 parts by weight based on weight % solids of the silicon-containing pressure sensitive adhesive (I), and the silicon-containing capping agent (ii) is present in the silicon-containing pressure sensitive adhesive (I) in an amount of from 0.1 to 15, alternatively 0.2 to 10 parts by weight based on weight % solids of the silicon-containing pressure sensitive adhesive (I).

The silicon-containing capping agent (ii) may be introduced to react with the pressure sensitive adhesive (i) after the pressure sensitive adhesive (i) has already been formed, i.e., after the silicone resin (a) and the polydiorganosiloxane (b) which make up the pressure sensitive adhesive (i) have condensation reacted.

Alternatively, the silicon-containing capping agent (ii) may be reacted in situ with the silicone resin (a) and the polydiorganosiloxane (b) such that the silicon-containing capping agent (ii) is present as the silicone resin (a) and the polydiorganosiloxane (b) are reacting to form the pressure sensitive adhesive (i). That is, in this in situ scenario, the silicon-containing capping agent (ii) is introduced either prior to or during the reaction of the silicone resin (a) and the polydiorganosiloxane (b) to form the pressure sensitive adhesive (i).

The silicon-containing capping agent (ii) is selected from the group of acrylate functional silanes, acrylate functional silazanes, acrylate functional disilazanes, acrylate functional disiloxanes, methacrylate functional silanes, methacrylate functional silazanes, methacrylate functional disilazanes, methacrylate functional disiloxanes, and combinations thereof.

The silicon-containing capping agent (ii) may be described to be of the general formula (XYR₂Si)₂D wherein X is a monovalent radical of the general formula A′E- where E is —O— or —NH— and A′ is an acryl group or a methacryl group, Y is a divalent alkylene radical having from 1 to 6 carbon atoms, R is a methyl or a phenyl radical, and D is a divalent or a trivalent organic hydrolyzable radical. Alternatively, D is —O— or —NH—. This particular silicon-containing capping agent (ii) may be selected from the group of Bis(3-methacryloxypropyl)tetramethyldisilazane, Bis(3-acryloxypropyl)tetramethyldisilazane, Bis(3-methacryloxypropyl)tetramethyldisiloxane, Bis(3-acryloxypropyl)tetramethyldisiloxane, and combinations thereof.

Alternatively, the silicon-containing capping agent (ii) may be described to be of the general formula XYR′_{b}SiZ_{3-b} wherein R′ is a methyl or a phenyl radical, Z is a monovalent hydrolyzable organic radical or a halogen, and b is 0, 1, or 2. Alternatively, the monovalent hydrolyzable organic radical is of the general formula R″O— where R″ is an alkylene radical. This particular silicon-containing capping agent (ii) may be selected from the group of 3-methacryloxypropyldimethylchlorosilane, 3-methacryloxypropyldichlorosilane, 3-methacryloxypropyltrichlorosilane, 3-methacryloxypropyldimethylmethoxysilane, 3-methacryloxypropylmethyldimethoxysilane, 3-methacryloxypropyltrimethoxysilane, 3-methacryloxypropyldimethylethoxysilane, 3-methacryloxypropylmethyldiethoxysilane, 3-methacryloxypropyltriethoxysilane, (methacryloxymethyl)dimethylmethoxysilane, (methacryloxymethyl)methyldimethoxysilane, (methacryloxymethyl)trimethoxysilane, (methacryloxymethyl)dimethylethoxysilane, (methacryloxymethyl)methyldiethoxysilane, methacryloxymethyltriethoxysilane, methacryloxypropyltriisopropoxysilane, 3-methacryloxypropyldimethylsilazane, 3-acryloxypropyldimethylchlorosilane, 3-acryloxypropyldichlorosilane, 3-acryloxypropyltrichlorosilane, 3-acryloxypropyldimethylmethoxysilane, 3-acryloxypropylmethyldimethoxysilane, 3-acryloxypropyltrimethoxysilane, 3-acryloxypropyldimethylsilazane, and combinations thereof.

A second silicon-containing capping agent (iii) may be used in conjunction with the silicon-containing capping agent (ii). This second silicon-containing capping agent (iii) is distinguishable from the silicon-containing capping agent (ii) in that the second silicon-containing capping agent (iii) is free of acrylate and methacrylate functionality and is capable of generating an endblocking triorganosilyl unit. If included, the second silicon-containing capping agent (iii) contributes to the reaction forming the silicon-containing pressure sensitive adhesive (I), along with the silicon-containing capping agent (ii) and the pressure sensitive adhesive (i). Suitable second silicon-containing capping agents (iii) include, but are not limited to, those described in U.S. Pat. No. 6,337,086 to Kanios et al., the disclosure of which has already been incorporated by reference for its teaching of these silicon-containing capping agents (iii).

The ethylenically unsaturated monomer (II) is a reactant that, along with the silicon-containing pressure sensitive adhesive (I) and the initiator (III), reacts to form the silicone acrylate hybrid composition.

The ethylenically unsaturated monomer (II) is present in the silicone acrylate hybrid composition in an amount of from 5 to 95, alternatively from 25 to 75, parts by weight based on 100 parts by weight of the silicone acrylate hybrid composition.

The ethylenically unsaturated monomer (II) has a glass transition temperature above room temperature (25° C.), alternatively above 35° C., alternatively above and including 40° C.

The ethylenically unsaturated monomer (II) may be any monomer having at least one carbon-carbon double bond. The ethylenically unsaturated monomer (II) used in the present invention may be a compound selected from the group of aliphatic acrylates, aliphatic methacrylates, cycloaliphatic acrylates, cycloaliphatic methacrylates, and combinations thereof. It is to be understood that each of the compounds, the aliphatic acrylates, the aliphatic methacrylates, the cycloaliphatic acrylates, and the cycloaliphatic methacrylates, include an alkyl radical which may include up to 20 carbon atoms.

The aliphatic acrylates that may be selected as one of the ethylenically unsaturated monomers (II) are selected from the group consisting of methyl acrylate, ethyl acrylate, propyl acrylate, n-butyl acrylate, iso-butyl acrylate, tert-butyl acrylate, hexyl acrylate, 2-ethylhexyl acrylate, iso-octyl acrylate, iso-nonyl acrylate, iso-pentyl acrylate, tridecyl acrylate, stearyl acrylate, lauryl acrylate, and mixtures thereof. The aliphatic methacrylates that may be selected as one of the ethylenically unsaturated monomers are selected from the group consisting of methyl methacrylate, ethyl methacrylate, propyl methacrylate, n-butyl methacrylate, iso-butyl methacrylate, tert-butyl methacrylate, hexyl methacrylate, 2-ethylhexyl methacrylate, iso-octyl methacrylate, iso-nonyl methacrylate, iso-pentyl methacrylate, tridecyl methacrylate, stearyl methacrylate, lauryl methacrylate, and mixtures thereof. The cycloaliphatic acrylate that may be selected as one of the ethylenically unsaturated monomers is cyclohexyl acrylate, and the cycloaliphatic methacrylate that may be selected as one of the ethylenically unsaturated monomers is cyclohexyl methacrylate. Styrene may also be selected.

Certain other monomers, described herein as polar monomers, may be used as the ethylenically unsaturated monomer (II) and may include supplemental functionality such as hydroxyl functionality. A polar monomer as used herein is an acrylic or methacrylic monomer having at least one polar group such as hydroxyl, alkoxy, amino, and alkenyl heterocycles. Examples of these polar monomers that are useful in the present invention include, but are not limited to, hydrophilic ethylenically unsaturated monomers of an amphoteric, anionic, cationic or anionic nature which are polymerizable by radical polymerization. More specific examples of these polar monomers include, but are not limited to, acrylic acid, methacrylic acid, itaconic acid, vinyl acetic acid, hydroxy ethyl acrylate, hydroxy ethyl methacrylate, hydroxy propyl acrylate, hydroxy propyl methacrylate, methoxyethyl acrylate, methoxyethyl methacrylate, aminoethyl acrylate, aminoethyl methacrylate, 2-N,N,N-trimethylammonium ethyl acrylate, 2-N,N,N-trimethylammonium ethyl methacrylate, acrylonitrile, methacrylonitrile, N,N-dimethylacrylamide, N-t-butylacrylamide, acrylamide, N-vinyl pyrrolidone, 2-acrylamido-2-methylpropane sulphonic acid, or salts thereof and the like.

The ethylenically unsaturated monomer (II) and the silicon-containing pressure sensitive adhesive (I) are polymerized in the presence of the initiator (III). The polymerization of the ethylenically unsaturated monomer (II) and the silicon-containing pressure sensitive adhesive (I) in the presence of the initiator (III) may be conducted at a temperature of from 50 to 100° C., alternatively of from 65 to 90° C.

The method of making the silicone acrylate hybrid composition may be employed in a batch process, semi-continuous process, or continuous process.

Although not required, the silicon-containing pressure sensitive adhesive (I), the ethylenically unsaturated monomer (II), and the initiator (III) may be mixed to form a pre-reaction mixture prior to the step of polymerizing and this pre-reaction mixture may be combined with a solvent prior to the step of polymerization. If these optional steps are conducted, then the polymerization obviously occurs with the components in the pre-reaction mixture after the pre-reaction mixture has been combined with the solvent.

It is to be understood that there are many different initiation mechanisms contemplated for use in the present invention to initiate the polymerization of the silicon-containing pressure sensitive adhesive (I) and the ethylenically unsaturated monomer (II). However, the typical initiator (III) is that known throughout the art as a free radical initiator. Free radical initiators include peroxides, azo compounds, redoxinitiators, and photo-initiators. The typical free radical initiators for application in the present invention are selected from the group of peroxides, azo compounds, and combinations thereof.

The initiator (III) is present in the silicone acrylate hybrid composition in an amount of from 0.005 to 3, alternatively from 0.01 to 2, parts by weight based on 100 parts by weight of the hybrid composition.

During the polymerization of the ethylenically unsaturated monomer (II) and the silicon-containing pressure sensitive adhesive (I), the silicone to acrylic ratio may be sufficiently controlled and optimized as desired. Controlling the silicone to acrylic ratio is desirable because the silicone acrylate hybrid composition may be optimized dependent on the end application for the silicone acrylate hybrid composition. An illustrative example of a mechanism is the rate controlled addition of the ethylenically unsaturated monomer or monomers (II) to the silicon-containing pressure sensitive adhesive (I). In certain applications, it may be desirable to have the silicone-based sub-species, or the overall silicone content, to exceed the acrylate-based sub-species, or the overall acrylic content. In other applications, it may be desirable for the opposite to be true. Independent of the end application, it is typical that the silicon-containing pressure sensitive adhesive (I) is present in the silicone acrylate hybrid composition in an amount of from 5 to 95, alternatively from 25 to 75, parts by weight based on 100 parts by weight of the silicone acrylate hybrid composition.

A polymerization solvent may be used during the polymerization to make the silicone acrylate hybrid composition to decrease the viscosity of the reaction mixture which allows for adequate mixing and heat transfer. The polymerization solvent may be any suitable material which is inert to the reaction ingredients and does not interfere with the reaction itself. Suitable polymerization solvents include, but are not limited to, aliphatic hydrocarbons such as hexane and heptane; alcohols such as methanol, ethanol and butanol; ketones such as acetone, methyl ethyl ketone and methyl isobutyl ketone; esters such as ethyl acetate, n-butyl acetate and i-butyl acetate; low viscosity silicone oils with linear, cyclic or branched structures which have a boiling point below 250° C. and a viscosity below 100 centipoises such as octamethylcyclotetrasiloxane, decamethylcyclopentasiloxane and hexamethyldisiloxane; and mixtures of two or more of the above mentioned polymerization solvents. If utilized, the amount of polymerization solvent is alternatively present in an amount of from 30 to 95, alternatively 40 to 70, parts by weight based on the total amount of the reactants and polymerization solvent.

The polymerization may occur in emulsion and the resulting silicone acrylate hybrid composition forming a silicone-in-water emulsion. The silicon-containing pressure sensitive adhesive (I), the ethylenically unsaturated monomer (II), and the initiator (III) may be mixed together and then emulsified to form a pre-reaction mixture prior to the step of polymerizing; they may be combined with a polymerization solvent prior to the step of emulsification. If these optional steps are conducted, then the polymerization occurs with the components in the pre-reaction mixture after the pre-reaction mixture has been emulsified in water. The emulsion may be obtained by various emulsification techniques known by persons of ordinary skill in the art such as but not limited to mechanical emulsion, twin screw extrusion, emulsion stabilized with emulsifier, surfactant or thickener, and pickering emulsion.

For cases where the molecular weight of the polymerization is to be controlled or limited, a chain transfer agent may be used. Chain transfer agents are known in the art and may include mercaptans, such as 1-butanethiol and dodecanethiol. If utilized, the amount of the chain transfer agent is alternatively from about 0 to 0.5 parts by weight per 100 parts by weight of the silicone acrylate hybrid composition.

Pressure sensitive adhesives usually are used with a release liner and a backing layer. The release liner is supporting the pressure sensitive adhesive until use, where the release liner is removed and the pressure sensitive adhesive is applied to adhere to a substrate. The backing layer is topping the surface of the pressure sensitive adhesive, facing outwards when the system is applied on the substrate. The backing layer may be preventing the drying out of the pressure sensitive adhesive or protecting it from wear off or preventing the external surface of the pressure sensitive adhesive to adhere to other substrates like sheets or clothes of a wearer. When used in transdermal drug delivery system, an additional drug containing layer may be present between the pressure sensitive adhesive and the backing layer. In other instances, the drug may also be present in the pressure sensitive adhesive layer.

Such pressure sensitive adhesives may be peeled off of the substrate and potentially repositioned.

The present non adhesive film forming silicone acrylate hybrid compositions do not require the use of a release liner or a backing layer. The topical composition comprising the non adhesive film forming silicone acrylate hybrid composition does not require the use of a release liner or a backing layer either. Once applied on a substrate, the non adhesive film forming silicone acrylate hybrid composition and/or topical composition containing it may be removed by rubbing off, wearing off, washing or wiping. Once removed, they may not be positioned again.

The non adhesive film forming silicone acrylate hybrid composition is characterized by a glass transition temperature, above that of a pressure sensitive adhesive silicone acrylate hybrid composition as per PCT/US2007/013321.

Glass transition temperature is of fundamental concern when considering adhesion, cohesion, and other properties of polymers. Pressure sensitive adhesives are characterized by a glass transition temperature below room temperature, and alternatively below 0° C. They are functioning as pressure sensitive adhesives when used above their glass transition temperature. When used below their glass transition temperature, they will become brittle and lose their adhesive potential. Traditionally, pressure sensitive adhesives are used at a temperature above their glass transition temperature.

The non adhesive film forming silicone acrylate hybrid composition may have multiple glass transition temperatures. The non adhesive film forming silicone acrylate hybrid compositions may be used at temperatures above, below or equal to the glass transition temperature. They may be used at any temperature without modifying their non adhesive film forming properties.

The non adhesive film forming silicone acrylate hybrid composition is used in conjunction with at least one physiologically acceptable ingredient and at least one physiologically acceptable solvent to form the topical compositions.

The at least one physiologically acceptable ingredient may be chosen from common cosmetic and dermatological ingredients such as emollients, waxes, moisturizers, surface active materials such as surfactants or detergents or emulsifiers, sebum absorbents or sebum control agents, vegetable or botanical extracts, pigments, colorants, thickeners, physiologically acceptable solvents, UV absorbers and sunscreen agents, preservatives, anti-dandruff agents, vitamins, proteins and amino-acid and their derivatives, nail care ingredients, fragrances, pH controlling agents, skin protectants, therapeutic active agents, anti acne agents, antifungal agents, antimicrobial agents, antioxidants, oxidizing agents, reducing agents, external analgesics, skin bleaching agents, anti-cancer agents, proteolytic enzymes, antihistamine, sedatives.

Examples of emollients include volatile or non-volatile silicone oils; silicone resins such as polypropylsilsesquioxane and phenyl trimethicone; silicone elastomers such as dimethicone crosspolymer; alkylmethylsiloxanes such as C30-45 Alkyl Methicone; silicone gums; organomodified silicone oils; volatile or non-volatile hydrocarbon compounds such as squalene, paraffin oils, petrolatum oils and naphthalene oils; hydrogenated or partially hydrogenated polyisobutene; isoeicosane; squalane; isoparaffin; isododecane; isodecane or isohexa-decane; branched C8-Ci6 esters; isohexyl neopentanoate; ester oils such as isononyl isononanoate, cetostearyl octanoate, isopropyl myristate, palmitate derivatives, stearates derivatives, isostearyl isostearate and the heptanoates, octanoates, decanoates or ricinoleates of alcohols or of polyalcohols, or mixtures thereof; hydrocarbon oils of plant origin, such as wheatgerm, sunflower, grapeseed, castor, shea, avocado, olive, soybean, sweet almond, palm, rapeseed, cotton seed, hazelnut, macadamia, jojoba, blackcurrant, evening primrose; or triglycerides of caprylic/capric acids; higher fatty acids, such as oleic acid, linoleic acid or linolenic acid.

Example of waxes include hydrocarbon waxes such as beeswax, lanolin wax, rice wax, carnauba wax, candelilla wax, microcrystalline waxes, paraffins, ozokerite, polyethylene waxes.

Examples of moisturizers include lower molecular weight aliphatic diols such as propylene glycol and butylene glycol; polyols such as glycerine and sorbitol; and polyoxyethylene polymers such as polyethylene glycol 200; hyaluronic acid and its derivatives.

Examples of surface active materials or emulsifiers may be anionic, cationic or non ionic, and include organomodified silicones such as dimethicone copolyol; oxyethylenated and/or oxypropylenated ethers of glycerol; oxyethylenated and/or oxypropylenated ethers of fatty alcohols such as ceteareth-30, C12-15 pareth-7; fatty acid esters of polyethylene glycol such as PEG-50 stearate, PEG-40 monostearate; saccharide esters and ethers, such as sucrose stearate, sucrose cocoate and sorbitan stearate, and mixtures thereof; phosphoric esters and salts thereof, such as DEA oleth-10 phosphate; sulphosuccinates such as disodium PEG-5 citrate lauryl sulphosuccinate and disodium ricinoleamido MEA sulphosuccinate; alkyl ether sulphates, such as sodium lauryl ether sulphate; isethionates; betaine derivatives.

Examples of sebum absorbents or sebum control agents include silica silylate, silica dimethyl silylate, dimethicone/vinyl dimethicone crosspolymer, polymethyl methacrylate, cross-linked methylmethacrylate and aluminum starch octenylsuccinate.

Examples of vegetable or botanical extracts are derived from plants (herbs, roots, flowers, fruits, or seeds) in oil or water soluble form, such as coconut, green tea, white tea, black tea, horsetail, sunflower, wheat germ, olive, grape, pomegranate, apricot, carrot, tomato, tobacco, bean, potato, actzuki bean, catechu, orange, cucumber, avocado, watermelon, banana, lemon or palm. Examples of herbal extracts include dill, horseradish, oats, neem, beet, broccoli, tea, pumpkin, soybean, barley, walnut, flax, ginseng, poppy, avocado, pea or sesame.

Examples of pigments and colorants include surface treated or untreated iron oxides, surface treated or untreated titanium dioxide, surface treated or untreated mica, silver oxide, silicates, chromium oxides, carotenoids, carbon black, chlorophyllin derivatives and yellow ocher.

Examples of thickeners include acrylamide copolymers, acrylate copolymers and salts thereof, xanthan gum and derivatives, cellulose gum and cellulose derivatives, carbomer, cassia gum, guar gum, cocamide derivatives, alkyl alcohols, gelatin, PEG-derivatives.

Examples of physiologically acceptable solvents include water; cyclic siloxanes such as octamethylcyclotetrasiloxane, decamethylcyclopentasiloxane, dodecamethylcyclohexasiloxane; linear siloxanes such as hexamethyldisiloxane, octamethyltrisiloxane, decamethyltetrasiloxane, dodecamethylpentasiloxane and dimethicone; hydrocarbon oils such as isododecane, isohexadecane, mineral oil, sunflower oil; ethanol; isopropyl alcohol; caprylic/capric triglyceride and mixtures thereof.

UV absorbers and sunscreen agents include those which absorb ultraviolet light between about 290-320 nanometers (the UV-B region) and those which absorb ultraviolet light in the range of 320-400 nanometers (the UV-A region).

Some examples of sunscreen agents are aminobenzoic acid, cinoxate, diethanolamine methoxycinnamate, digalloyl trioleate, dioxybenzone, ethyl 4-[bis(Hydroxypropyl)]aminobenzoate, glyceryl aminobenzoate, homosalate, lawsone with dihydroxyacetone, menthyl anthranilate, octocrylene, ethyl hexyl methoxycinnamate, octyl salicylate, oxybenzone, padimate O, phenylbenzimidazole sulfonic acid, red petrolatum, sulisobenzone, titanium dioxide, and trolamine salicylate.

Some examples of UV absorbers are acetaminosalol, allatoin PABA, benzalphthalide, benzophenone, benzophenone 1-12, 3-benzylidene camphor, benzylidenecamphor hydrolyzed collagen sulfonamide, benzylidene camphor sulfonic Acid, benzyl salicylate, bornelone, bumetriozole, butyl Methoxydibenzoylmethane, butyl PABA, ceria/silica, ceria/silica talc, cinoxate, DEA-methoxycinnamate, dibenzoxazol naphthalene, di-t-butyl hydroxybenzylidene camphor, digalloyl trioleate, diisopropyl methyl cinnamate, dimethyl PABA ethyl cetearyldimonium tosylate, dioctyl butamido triazone, diphenyl carbomethoxy acetoxy naphthopyran, disodium bisethylphenyl tiamminotriazine stilbenedisulfonate, disodium distyrylbiphenyl triaminotriazine stilbenedisulfonate, disodium distyrylbiphenyl disulfonate, drometrizole, drometrizole trisiloxane, ethyl dihydroxypropyl PABA, ethyl diisopropylcinnamate, ethyl methoxycinnamate, ethyl PABA, ethyl urocanate, etrocrylene ferulic acid, glyceryl octanoate dimethoxycinnamate, glyceryl PABA, glycol salicylate, homosalate, isoamyl p-methoxycinnamate, isopropylbenzyl salicylate, isopropyl dibenzolylmethane, isopropyl methoxycinnamate, menthyl anthranilate, menthyl salicylate, 4-methylbenzylidene, camphor, octocrylene, octrizole, octyl dimethyl PABA, ethyl hexyl methoxycinnamate, octyl salicylate, octyl triazone, PABA, PEG-25 PABA, pentyl dimethyl PABA, phenylbenzimidazole sulfonic acid, polyacrylamidomethyl benzylidene camphor, potassium methoxycinnamate, potassium phenylbenzimidazole sulfonate, red petrolatum, sodium phenylbenzimidazole sulfonate, sodium urocanate, TEA-phenylbenzimidazole sulfonate, TEA-salicylate, terephthalylidene dicamphor sulfonic acid, titanium dioxide, triPABA panthenol, urocanic acid, and VA/crotonates/methacryloxybenzophenone-1 copolymer.

Example of preservatives include paraben derivatives, hydantoin derivatives, chlorhexidine and its derivatives, imidazolidinyl urea, phenoxyethanol, silver derivatives, salicylate derivatives, triclosan, zinc pyrithione and mixtures thereof.

Examples of antidandruff agents include pyridinethione salts, selenium compounds such as selenium disulfide, and soluble antidandruff agents.

Examples of vitamins include a variety of different organic compounds such as alcohols, acids, sterols, and quinones. They may be classified into two solubility groups: lipid-soluble vitamins and water-soluble vitamins. Lipid-soluble vitamins that have utility in personal care formulations include retinol (vitamin A), ergocalciferol (vitamin D2), cholecalciferol (vitamin D3), phytonadione (vitamin K1), and tocopherol (vitamin E). Water-soluble vitamins that have utility in personal care formulations include ascorbic acid (vitamin C), thiamin (vitamin B1) niacin (nicotinic acid), niacinamide (vitamin B3), riboflavin (vitamin B2), pantothenic acid (vitamin B5), biotin, folic acid, pyridoxine (vitamin B6), and cyanocobalamin (vitamin B12). Additional examples of vitamins include derivatives of vitamins such as retinyl palmitate (vitamin A palmitate), retinyl acetate (vitamin A acetate), retinyl linoleate (vitamin A linoleate), and retinyl propionate (vitamin A propionate), tocopheryl acetate (vitamin E acetate), tocopheryl linoleate (vitamin E linoleate), tocopheryl succinate (vitamin E succinate), tocophereth-5, tocophereth-10, tocophereth-12, tocophereth-18, tocophereth-50 (ethoxylated vitamin E derivatives), PPG-2 tocophereth-5, PPG-5 tocophereth-2, PPG-10 tocophereth-30, PPG-20 tocophereth-50, PPG-30 tocophereth-70, PPG-70 tocophereth-100 (propoxylated and ethoxylated vitamin E derivatives), sodium tocopheryl phosphate, ascorbyl palmitate, ascorbyl dipalmitate, ascorbyl glucoside, ascorbyl tetraisopalmitate, tetrahexadecyl ascorbate, ascorbyl tocopheryl maleate, potassium ascorbyl tocopheryl phosphate or tocopheryl nicotinate.

Proteins or amino-acids and their derivatives include those extracted from wheat, soy, rice, corn, keratin, elastin or silk. Most proteins are in the hydrolyzed form and they may also be quaternized.

Example of nail care ingredients include butyl acetate; ethyl acetate; nitrocellulose; acetyl tributyl citrate; isopropyl alcohol; adipic acid/neopentyl glycol/trimelitic anhydride copolymer; stearalkonium bentonite; acrylates copolymer; calcium pantothenate; Cetraria islandica extract; Chondrus crispus; styrene/acrylates copolymer; trimethylpentanediyl dibenzoate-1; polyvinyl butyral; N-butyl alcohol; propylene glycol; butylene glycol; mica; silica; tin oxide; calcium borosilicate; synthetic fluorphlogopite; polyethylene terephtalate; sorbitan laurate derivatives; talc; jojoba extract; diamond powder; isobutylphenoxy epoxy resin; silk powder.

Examples of fragrances or perfume include hexyl cinnamic aldehyde; anisaldehyde; methyl-2-n-hexyl-3-oxo-cyclopentane carboxylate; dodecalactone gamma; methylphenylcarbinyl acetate; 4-acetyl-6-tert-butyl-1,1-dimethyl indane; patchouli; olibanum resinoid; labdanum; vetivert; copaiba balsam; fir balsam; 4-(4-hydroxy-4-methyl pentyl)-3-cyclohexene-1-carboxaldehyde; methyl anthranilate; geraniol; geranyl acetate; linalool; citronellol; terpinyl acetate; benzyl salicylate; 2-methyl-3-(p-isopropylphenyl)-propanal; phenoxyethyl isobutyrate; cedryl acetal; aubepine; musk fragrances; macrocyclic ketones; macrolactone musk fragrances; ethylene brassylate.

Examples of pH controlling agents include any water soluble acid such as a carboxylic acid or a mineral acid such as hydrochloric acid, sulphuric acid, and phosphoric acid, monocarboxylic acid such as acetic acid and lactic acid, and polycarboxylic acids such as succinic acid, adipic acid, and citric acid.

Some examples of skin protectants are allantoin, aluminium acetate, aluminium hydroxide, aluminium sulfate, calamine, cocoa butter, cod liver oil, colloidal oatmeal, dimethicone, glycerin, kaolin, lanolin, mineral oil, petrolatum, shark liver oil, sodium bicarbonate, talc, witch hazel, zinc acetate, zinc carbonate, and zinc oxide.

Examples of therapeutic active agents include anti acne agents, antifungal agents, antimicrobial agents, external analgesic, skin bleaching agents, anticancer agents, proteolytic enzymes, antihistamine, sedatives, antibiotic, antiseptic, antibacterial, anti-inflammatory, astringents, hormones, smoking cessation compositions, tranquillizer, anticonvulsant, anticoagulant agents, healing factors, cell growth nutrients.

Specific examples of suitable therapeutic active agents include penicillins, cephalosporins, tetracyclines, macrolides, epinephrine, amphetamines, aspirin, acetominophen, barbiturates, catecholamines, benzodiazepine, thiopental, codeine, morphine, procaine, lidocaine, benzocaine, sulphonamides, ticonazole, perbuterol, furosamide, prazosin, prostaglandins, salbutamol, indomethicane, diclofenac, glafenine, dipyridamole, theophylline and retinol.

Some examples of anti acne agents are salicylic acid and sulfur.

Some examples of antifungal agents are calcium undecylenate, undecylenic acid, zinc undecylenate, and povidone-iodine.

Some examples of antimicrobial agents are alcohol, benzalkonium chloride, benzethonium chloride, hydrogen peroxide, methylbenzethonium chloride, phenol, poloxamer 188, and povidone-iodine.

Some examples of antioxidants are acetyl cysteine, arbutin, ascorbic acid, ascorbic acid polypeptide, ascorbyl dipalmitate, ascorbyl methylsilanol pectinate, ascorbyl palmitate, ascorbyl stearate, BHA, p-hydroxyanisole, BHT, t-butyl hydroquinone, caffeic acid, Camellia sinensis Oil, chitosan ascorbate, chitosan glycolate, chitosan salicylate, chlorogenic acids, cysteine, cysteine HCl, decyl mercaptomethylimidazole, erythorbic acid, diamylhydroquinone, di-t-butylhydroquinone, dicetyl thiodipropionate, dicyclopentadiene/t-butylcresol copolymer, digalloyl trioleate, dilauryl thiodipropionate, dimyristyl thiodipropionate, dioleyl tocopheryl methylsilanol, isoquercitrin, diosmine, disodium ascorbyl sulfate, disodium rutinyl disulfate, distearyl thiodipropionate, ditridecyl thiodipropionate, dodecyl gallate, ethyl ferulate, ferulic acid, hydroquinone, hydroxylamine HCl, hydroxylamine sulfate, isooctyl thioglycolate, kojic acid, madecassicoside, magnesium ascorbate, magnesium ascorbyl phosphate, melatonin, methoxy-PEG-7 rutinyl succinate, methylene di-t-butylcresol, methylsilanol ascorbate, nordihydroguaiaretic acid, octyl gallate, phenylthioglycolic acid, phloroglucinol, potassium ascorbyl tocopheryl phosphate, thiodiglycolamide, potassium sulfite, propyl gallate, rosmarinic acid, rutin, sodium ascorbate, sodium ascorbyl/cholesteryl phosphate, sodium bisulfite, sodium erythorbate, sodium metabisulfide, sodium sulfite, sodium thioglycolate, sorbityl furfural, tea tree (Melaleuca aftemifolia) oil, tocopheryl acetate, tetrahexyldecyl ascorbate, tetrahydrodiferuloylmethane, tocopheryl linoleate/oleate, thiodiglycol, tocopheryl succinate, thiodiglycolic acid, thioglycolic acid, thiolactic acid, thiosalicylic acid, thiotaurine, retinol, tocophereth-5, tocophereth-10, tocophereth-12, tocophereth-18, tocophereth-50, tocopherol, tocophersolan, tocopheryl linoleate, tocopheryl nicotinate, tocoquinone, o-tolyl biguanide, tris(nonylphenyl)phosphite, ubiquinone, and zinc dibutyldithiocarbamate.

Some examples of oxidizing agents are ammonium persulfate, calcium peroxide, hydrogen peroxide, magnesium peroxide, melamine peroxide, potassium bromate, potassium caroate, potassium chlorate, potassium persulfate, sodium bromate, sodium carbonate peroxide, sodium chlorate, sodium iodate, sodium perborate, sodium persulfate, strontium dioxide, strontium peroxide, urea peroxide, and zinc peroxide.

Some examples of reducing agents are ammonium bisufite, ammonium sulfite, ammonium thioglycolate, ammonium thiolactate, cystemaine HCl, cystein, cysteine HCl, ethanolamine thioglycolate, glutathione, glyceryl thioglycolate, glyceryl thioproprionate, hydroquinone, p-hydroxyanisole, isooctyl thioglycolate, magnesium thioglycolate, mercaptopropionic acid, potassium metabisulfite, potassium sulfite, potassium thioglycolate, sodium bisulfite, sodium hydrosulfite, sodium hydroxymethane sulfonate, sodium metabisulfite, sodium sulfite, sodium thioglycolate, strontium thioglycolate, superoxide dismutase, thioglycerin, thioglycolic acid, thiolactic acid, thiosalicylic acid, and zinc formaldehyde sulfoxylate.

Some examples of external analgesics are benzyl alcohol, capsicum oleoresin (Capsicum frutescens oleoresin), methyl salicylate, camphor, phenol, capressure sensitive adhesiveicin, juniper tar (Juniperus oxycedrus tar), phenolate sodium (sodium phenoxide), capsicum (Capsicum frutescens), menthol, resorcinol, methyl nicotinate, and turpentine oil (turpentine).

An example of a skin bleaching agent is hydroquinone.

Examples of anti-cancer agents include alkylating agents (such as busulfan, fluorodopan), antimitotic agents (such as colchicine, rhizoxin), topoisomerase I inhibitors (such as camptothecin and its derivatives), topoisomerase II inhibitors (such as menogaril, amonafide), RNA/DNA or DNA anti-metabolites (such as acivicin, guuanazole), plant alkaloids and terpenoids, antineoplastics and some plant-derived compounds (such as podophyllotoxin, vinca alkaloids).

Examples of proteolytic enzymes include nattokinase, serratiopeptidase, bromelain, papain.

Examples of antihistamine or H1 histamine blockers include brompheniramine, clemastine, cetirizine, loratadine, fexofenadine.

Examples of sedatives include barbiturates (such as phenobarbitol), benzodiazepines (such as lorazepam), herbal sedatives, benzodiazepine-like drugs (such as zolpidem, zopiclone).

Further materials suitable for topical compositions are well known to the person skilled in the art and are described in many text books as well as other publications.

The topical composition may be a cosmetic or dermatological composition, i.e., a composition that is compatible with keratin materials such as the skin, mucous membranes, the hair, the eyelashes, the eyebrows and the nails.

The topical compositions include creams, ointments, hydrogels, onguents, lotions, mousses, foams, sticks, sprays, masks, makeup compositions, nailcare products, sun protection compositions, cleansing compositions. The topical compositions herein are generally acceptable on many keratinous substrates, such as skin, hair or nails.

The general level of non adhesive film forming silicone acrylate hybrid composition in the topical compositions may vary from 1% to 25% by weight, alternatively from 5% to 20%, alternatively from 10% to 20%. The compositions are prepared by mixing the non adhesive film forming silicone acrylate hybrid composition with other compatible oil based ingredients to form an oil phase of the composition, potentially heating. The composition may be anhydrous or may be processed as an emulsion.

The general level of the at least one physiologically acceptable ingredient may vary from 0.01% to 25% by weight, alternatively from 1% to 20%, alternatively from 1% to 15%.

The general level of the at least one physiologically acceptable solvent ingredient may vary from 30% to 98.99% by weight, alternatively from 40% to 95%.

When making an emulsion composed of an oil phase and a water phase, the emulsifiers may be added to the appropriate phase, and the oil and aqueous phases may then mixed together to form the final composition. Anhydrous emulsions of oil phases with non aqueous phases such as glycols may also be prepared.

When the non adhesive film forming silicone acrylate hybrid composition is in the form of an emulsion, it may be mixed with the ingredients of the aqueous phase and be processed further as appropriate.

The composition may be adjusted for pH. Sensitive ingredients may further be added as appropriate, such as fragrances, nacres. Mixing devices are those generally used by the man skilled in the art to prepare personal care compositions and include mixing vessels with paddles, stirrers, homogenisers, scrapers and other equipment which is known to the person skilled in the art. The compositions may be prepared at temperatures ranging from 15° C. to 90° C., alternatively from 20° C. to 60° C., or alternatively at room temperature (20° C.-25° C.).

The topical compositions may be applied by rubbing, painting, spraying, or any other conventional method of applying topical compositions on keratinous substrates.

The topical compositions may have varying degrees of permeability, from permeable to occlusive, depending on the ingredients used in conjunction with the non adhesive film forming silicone acrylate hybrid composition.

An ointment may comprise 60-90 wt % petrolatum, 5-15 wt % stearoxytrimethylsilane and stearyl alcohol and 5-15 wt % of non adhesive film forming silicone acrylate hybrid composition.

A cream may comprise 5-12 wt % silicone elastomer, 3-8 wt % dimethiconol, 5-20 wt % cyclopentasiloxane, 1-5 wt % silicone emulsifier, 50-80 wt % water, 0, 01-3 wt % hydrophilic drug and 5-15 wt % of non adhesive film forming silicone acrylate hybrid composition.

A spray may comprise 2-8 wt % silicone elastomer, 2-8 wt % dimethiconol, 75-85 wt % cyclopentasiloxane and 5-10 wt % of non adhesive film forming silicone acrylate hybrid composition.

A medicated topical film may comprise from 0.01% to 20% wt of an active and 80% to 99.9% other ingredients such as silicone polyether, dimethiconol, castor oil, isopropyl myristate, propylene glycol, glyceride derivative and 5-10 wt % of non adhesive film forming silicone acrylate hybrid composition.

The topical composition is applied on the surface of keratin materials.

### EXAMPLES

The following examples are included to demonstrate embodiments of the invention. It should be appreciated by those of skill in the art that the techniques disclosed in the examples which follow represent techniques discovered by the inventor to function well in the practice of the invention, and thus may be considered to constitute typical modes for its practice. However, those of skill in the art should, in light of the present disclosure, appreciate that many changes may be made in the specific embodiments which are disclosed and still obtain a like or similar result without departing from the spirit and scope of the invention. All percentages are in wt. %.

The following Examples illustrating specifics associated with the making of the non adhesive film forming silicone acrylate hybrid composition of the present invention, as presented herein, are intended to illustrate and not limit the invention.

The components used in the following examples are as follows:
EAS is a silicon-containing pressure sensitive adhesive composition and is 63.4% weight solids in ethyl acetate. EAS is produced through a condensation reaction of a silanol endblocked polydimethylsiloxane (PDMS) with a silicate resin and that is endblocked with a silicon-containing capping agent which provides the acrylate or methacrylate functionality.
PSA 1 is an uncapped silicone PSA that is produced through the condensation reaction between a hydroxy end-blocked polydimethylsiloxane (PDMS) with a viscosity of 50,000 cP and a hydroxy end-blocked silicate resin at a compositional ratio of 35% PDMS to 65% resin.
2-EHA is 2-ethylhexyl acrylate commercially available from Aldrich.
MA is methyl acrylate commercially available from Aldrich.
MMA is methyl methacrylate commercially available from Aldrich.
BA is butyl acrylate commercially available from Aldrich.
Vazo® 67 is 2,2′-azobis(methylbutyronitrile) commercially available from Dupont.

The glass transition temperatures (Tg) of a particular Example were determined by Differential Scanning calorimetry (DSC). Approximately 2 to 4 milligrams of dried material was loaded into a standard aluminum pan. The aluminum pan with sample was placed into the cell of a DSC. The sample was first cooled to <−150° C. and then heated to +150° C. at a rate of 10° C./minute under a helium purge. The individual Tg is reported as the half-height of the material transition. One material may have multiple thermal transitions based on its composition.

### Examples 1 to 3

### Example 1

131.29 g of MMA, 121.5 g of BA, 392.92 g of PSA 1, 65.48 g of ethyl acetate solvent and 0.365 g Vazo® 67 were added to form a pre-reaction mixture. The pre-reaction mixture was allowed to stir 15 minutes until thoroughly homogeneous. After mixing, 182.04 g of the pre-reaction mixture and 491.8 g of ethyl acetate solvent were added to a reactor. The remaining portion of the pre-reaction mixture was added to a separate reservoir. Heating and mixing was then begun on the mixture in the reactor. The reaction temperature was set at 78° C. As soon as the reaction temperature was achieved, the mixture was allowed to react for 60 minutes prior to adding more of the pre-reaction mixture to the reactor. Once 60 minutes elapsed, the mixture in the reservoir was added at a rate of 2.34 grams/minutes for 253 minutes until the mixture in the reservoir was finished. The mixture in the reactor was then reacted at 78° C. for an additional 340 minutes to form the silicone acrylate hybrid composition. Upon completion, the silicone acrylate hybrid composition was allowed to cool to room temperature before being removed from the reactor. The silicone acrylate hybrid composition was then dried in a forced air oven at 150° C. for 60 minutes to remove the ethyl acetate solvent. The final product was opaque in color. Example 1 is characterized by a Tg(1) of −119° C. and a Tg(2) of 46.6° C.

### Example 2

216.68 g of MMA, 35.34 g of BA, 393.17 g of PSA 1, 65.20 g of ethyl acetate solvent and 0.365 g Vazo® 67 were added to form a pre-reaction mixture. The pre-reaction mixture was allowed to stir 15 minutes until thoroughly homogeneous. After mixing, 171.0 g of the pre-reaction mixture and 491.0 g of ethyl acetate solvent were added to a reactor. The remaining portion of the pre-reaction mixture was added to a separate reservoir. Heating and mixing was then begun on the mixture in the reactor. The reaction temperature was set at 78° C. As soon as the reaction temperature was achieved, the mixture was allowed to react for 60 minutes prior to adding more of the pre-reaction mixture to the reactor. Once 60 minutes elapsed, the mixture in the reservoir was added at a rate of 2.40 grams/minutes for 225 minutes until the mixture in the reservoir was finished. The mixture in the reactor was reacted at 78° C. for an additional 375 minutes to form the silicone acrylate hybrid composition. Upon completion, the silicone acrylate hybrid composition was allowed to cool to room temperature before being removed from the reactor. The silicone acrylate hybrid composition was then dried in a forced air oven at 150° C. for 60 minutes to remove the ethyl acetate solvent. The final product was opaque in color. Example 2 is characterized by a Tg(1) of −118.7° C. and a Tg(2) of 104.2° C.

### Example 3

179.01 g of MMA, 73.87 g of BA, 392.99 g of PSA 1, 65.62 g of ethyl acetate solvent and 0.365 g Vazo® 67 were added to form a pre-reaction mixture. The pre-reaction mixture was allowed to stir 15 minutes until thoroughly homogeneous. After mixing, 181.0 g of the pre-reaction mixture and 487.0 g of ethyl acetate solvent were added to a reactor. The remaining portion of the pre-reaction mixture was added to a separate reservoir. Heating and mixing was then begun on the mixture in the reactor. The reaction temperature was set at 78° C. As soon as the reaction temperature was achieved, the mixture was allowed to react for 60 minutes prior to adding more of the pre-reaction mixture to the reactor. Once 60 minutes elapsed, the mixture in the reservoir was added at a rate of 2.30 grams/minutes for 230 minutes until the mixture in the reservoir was finished. The mixture in the reactor was reacted at 78° C. for an additional 355 minutes to form the silicone acrylate hybrid composition. Upon completion, the silicone acrylate hybrid composition was allowed to cool to room temperature before being removed from the reactor. The silicone acrylate hybrid composition was then dried in a forced air oven at 150° C. for 60 minutes to remove the ethyl acetate solvent. The final product was opaque in color. Example 3 is characterized by a Tg(1) of −118.5° C. and a Tg(2) of 79.2° C.

Topical compositions may be formed using Examples 1 to 3 in combination with various ingredients, such as petrolatum, mineral oil, caprylic/capric triglyceride, propanediol, ethanol, dicaprylyl ether.

A first topical composition was prepared as follows: first, a solution of silicone acrylate hybrid composition in ethyl acetate (14.5%-85.5% respectively) was formed; subsequently, the dissolved silicone acrylate hybrid composition were mixed with another ingredient to form a homogenous composition containing 13.8% silicone acrylate hybrid composition, 81.4% ethyl acetate and 4.8% other ingredient.

The topical compositions were then applied as films (on an aluminium cup) composed of 74.4% silicone acrylate hybrid composition and 25.6% other ingredient, after complete evaporation of the ethyl acetate. Resulting films may have varying texture, for example described as waxy, rigid, smooth or flexible—see Table 9.

TABLE 9

| | Silicone | |
|---|---|---|
| | acrylate | |
| Other | hybrid | |
| ingredient | composition | Composition texture |
| Petrolatum | Example 1 | Soft wax |
| | Example 2 | Soft wax |
| | Example 3 | Soft wax |
| Mineral oil | Example 1 | Rigid film |
| | Example 2 | Rigid film |
| | Example 3 | Rigid film |
| Caprylic/capric | Example 1 | Flexible film |
| triglyceride | Example 2 | Flexible film |
| | Example 3 | Flexible film |
| Ethanol | Example 1 | Smooth film |
| | Example 2 | Smooth film |
| | Example 3 | Smooth film |
| Dicaprylyl | Example 1 | Soft wax |
| ether | Example 2 | Soft wax |
| | Example 3 | Rigid film |

A second topical composition was prepared as follows: first, a solution of silicone acrylate hybrid composition in ethyl acetate (14.5%-85.5% respectively) was formed; subsequently, the dissolved silicone acrylate hybrid composition were mixed with another ingredient to form a homogenous composition containing 14.3% silicone acrylate hybrid composition, 84.1% ethyl acetate and 1.6% other ingredient.

The topical compositions were then applied as films (on an aluminium cup) composed of 90.0% silicone acrylate hybrid composition and 10.0% other ingredient, after complete evaporation of the ethyl acetate. Resulting films may have varying texture, for example described as waxy, rigid, smooth or flexible—see Table 10.

TABLE 10

| | Silicone | |
|---|---|---|
| | acrylate | |
| Other | hybrid | |
| ingredient | composition | Composition texture |
| Petrolatum | Example 1 | Rigid film |
| | Example 2 | Rigid film |
| | Example 3 | Rigid film |
| Caprylic/capric | Example 1 | Flexible film |
| triglyceride | Example 2 | Soft wax |
| | Example 3 | Waxy film |
| Propanediol | Example 1 | Rigid film |
| | Example 2 | Rigid film |
| | Example 3 | Smooth film |
| Dicaprylyl | Example 1 | Rigid film |
| ether | Example 2 | Rigid film |
| | Example 3 | Rigid film |

### Examples 4 to 6

Examples 4 to 6 were prepared as follows—with amounts listed in Table 11: in a first beaker, lidocaine was solubilised in caprylic/capric triglyceride, heated in a water bath to 60° C. for 15 min under magnetic stirring, then let to cool down; the solution of non adhesive film forming silicone acrylate hybrid composition in ethyl acetate was then added to the lidocaine solution, and let to homogenize on a roller mixing plate for 1 h.

TABLE 11

| | Silicone | | | | |
|---|---|---|---|---|---|
| | acrylate | | | Caprylic/ | |
| | hybrid | Ethyl | | Capric | Total |
| | composition | Acetate | Lidocaine | Triglyceride | amount |
| | (g) | (g) | (g) | (g) | (g) |
| Example 4 | 4.37 | 25.69 | 2.00 | 8.01 | 40.07 |
| Example 5 | 4.35 | 25.73 | 2.01 | 8.05 | 40.14 |
| Example 6 | 4.38 | 25.70 | 2.01 | 8.02 | 40.11 |

The Examples 4 to 6 were used to form films by applying a certain amount spread as a film of 1.016 mm (40 mils) thickness on a release liner from 3M reference 9956 (release coating bonded on a clear polyester film), film which is left under the fume hood at room temperature (about 22° C.) for 1 night. The evaporation of ethyl acetate may not be complete before the test. The use of a release liner for support to Examples 4-6 was to ensure formation of a smooth and homogeneous film in a reproducible way, not as support for an adhesive film.

Of the resulting films, 4 circular samples were taken of 14 mm diameter. Sample weights were measured as listed in Table 12.

The samples were then applied on dermatomed pig skin (750 μm) and skin penetration study was conducted, using Phosphate Buffered Saline (PBS—water based solution containing sodium chloride and sodium phosphate) as receptor fluid. Analysis of the receptor fluid was carried out via UPLC chromatography to quantify amounts of lidocaine passed through the skin.

The results—also in Table 12, show that the lidocaine is released from the Examples 4 to 6.

TABLE 12

| | Average on sample weight | Drug passed through the |
|---|---|---|
| Sample | (g/cm²) | skin (μg/cm²) |
| Example 4 | 0.0130 | 268.8 ± 107.1 |
| Example 5 | 0.0118 | 153.1 ± 13.8 |
| Example 6 | 0.0152 | 154.9 ± 52.9 |

1. A topical composition comprising a non adhesive film forming silicone acrylate hybrid composition and at least one physiologically acceptable ingredient and at least one physiologically acceptable solvent.

2. The topical composition of claim 1 where the non adhesive film forming silicone acrylate hybrid composition comprises the reaction product of:
I. a silicon-containing pressure sensitive adhesive composition comprising acrylate or methacrylate functionality
II. an ethylenically unsaturated monomer having a glass transition temperature above room temperature; selected from the group of aliphatic acrylates, aliphatic methacrylates, cycloaliphatic acrylates, cycloaliphatic methacrylates, and combinations thereof, each of said compounds having up to 20 carbon atoms in the alkyl radical, and
III. an initiator.

3. The topical composition as claimed in claim 1 wherein the topical composition is compatible with keratin materials.

4. The topical composition of claim 3 wherein the keratin materials include the skin, mucous membranes, the hair, the eyelashes, the eyebrows and the nails.

5. The topical composition as claimed in claim 1 wherein the at least one physiologically acceptable ingredient is selected from emollients, waxes, moisturizers, surface active materials such as surfactants or detergents or emulsifiers, sebum absorbents or sebum control agents, vegetable or botanical extracts, pigments, colorants, thickeners, physiologically acceptable solvents, UV absorbers and sunscreen agents, preservatives, anti-dandruff agents, vitamins, proteins and amino-acid and their derivatives, nail care ingredients, fragrances, pH controlling agents, skin protectants, therapeutic active agents, anti acne agents, antifungal agents, antimicrobial agents, antioxidants, oxidizing agents, reducing agents, external analgesics, skin bleaching agents, anti-cancer agents, proteolytic enzymes, antihistamine, sedatives.

6. The topical composition as claimed in claim 1 wherein the at least one physiologically acceptable solvent is selected from cyclic siloxanes such as octamethylcyclotetrasiloxane, decamethylcyclopentasiloxane, dodecamethylcyclohexasiloxane; linear siloxanes such as hexamethyldisiloxane, octamethyltrisiloxane, decamethyltetrasiloxane, dodecamethylpentasiloxane and dimethicone; hydrocarbon oils such as isododecane, isohexadecane, mineral oil, sunflower oil; ethanol; isopropyl alcohol; caprylic/capric triglyceride and mixtures thereof.

7. The topical composition as claimed in claim 1 where the non adhesive film forming silicone acrylate hybrid composition is present from 1% to 25% by weight, the at least one physiologically acceptable ingredient is present from 0.01% to 25% by weight and the at least one physiologically acceptable solvent is present from 30% to 98.99% by weight.

8. A keratin material having the topical composition as claimed in claim 1 applied to it.

9. A method of treating a keratin material comprising applying a topical composition comprising a non adhesive film forming silicone acrylate hybrid composition, at least one physiologically acceptable ingredient and at least one physiologically acceptable solvent.
